(19) United States
(12) Patent Application Publication
(10) Pub. No.: US 2024/0417665 A1
(43) Pub. Date: Dec. 19, 2024

(54) BIOREACTOR HEATING APPARATUS

(71) Applicant: Applikon Biotechnology B.V., Delft (NL)

(72) Inventors: Wilbert Timotheüs WALVOORT, Delft (NL); Menno Jan VASTENHOLT, Delft (NL)

(73) Assignee: Applikon Biotechnology B.V., Delft (NL)

(21) Appl. No.: 18/700,586

(22) PCT Filed: Oct. 3, 2022
(86) PCT No.: PCT/NL2022/050552

§ 371 Date: Apr. 11, 2024

(30) Foreign Application Priority Data
Oct. 11, 2021 (NL) 2029378

(51) Int. Cl. C12M 1/02; C12M 1/00; C12M 1/06

(52) U.S. Cl. CPC C12M 41/22; C12M 23/22; C12M 23/26; C12M 27/06

(57) ABSTRACT

A heating apparatus (10) for a bioreactor (70), wherein the heating apparatus has a transparent body (20) that allows for direct visualization of a bioreactor culture and is adapted to be removably coupled to and provide customized heating to one or more different types of bioreactor vessels (72).

### FIELD OF ART

This disclosure relates to a heating apparatus, bioreactor system and related methods for use. In particular, this disclosure addresses a device, system and method for efficiently heating and regulating the temperature of a bioreactor in order to facilitate growth and cultivation of microbes and other organisms.

### BACKGROUND

A bioreactor generally refers to a device or system used to support a biologically active environment. Bioreactors, may include a vessel in which a biological process is carried out, such as cultivation of organisms or biochemically active substances derived from such organisms. This process can be either aerobic or anaerobic. A bioreactor may also refer to a device or system designed to grow cells or tissues in the context of cell culture.

Bioreactors may be cylindrical, have variable diameters or other configurations and may range in size from milliliters to cubic meters. Bioreactors can be constructed from various materials, such as glass, stainless steel or polymers. The size and configuration of a bioreactor may be determined in part by its intended mode of operation, e.g., bioreactors configured for batch processing, fed-batch processing, continuous, multi-use processing or single-use bioprocessing activities. By way of example, continuous use bioreactors are often configured as autoclavable reusable, glass or stainless cylindrical vessels. Single-use bioreactors, by contrast, are disposable and often constructed from plastic or other polymers.

A typical bioreactor comprises the following parts: an agitator or impeller—used for the mixing of the contents of the reactor which keeps the “cells” in the perfect homogenous condition for better transport of nutrients and oxygen to the cells; a baffle-used to break the vortex formation in the vessel, which is usually highly undesirable as it changes the center of gravity of the system and consumes additional power without adding to the mixing; a sparger—in aerobic cultivation process, the purpose of the sparger is to supply adequate gasses (such as oxygen or CO₂) to the growing cells and a jacket-providing an annular area with circulating hot or cold water for heat transfer, alternatively, depending upon the application and bioreactor type/needs, Peltier heating elements or heating blankets potentially may be used.

The rate and quality of bioprocessing is directly dependent upon the environmental conditions and parameters established by the bioreactor, such as nutrient level, pH, O₂ levels, and temperature. Temperature regulation and maintenance, in particular, is a key factor in promoting the growth of organisms and production of biochemically active substances. The ability to adjust, regulate and customize the temperature of a bioreactor culture as well as the ability to transfer heat uniformly throughout the bioreactor culture substantially affects overall bioprocessing. Existing heaters for temperature regulation typically only provide a primary heating or cooling unit usually adjoining and supporting a distal base of a bioreactor vessel. Such devices do not provide uniform, consistent heat transfer throughout the culture medium, often creating temperature gradients across the bioreactor culture and limiting the rate of heat transfer. Pockets of the bioreactor culture may be hotter or colder than a desired temperature range when using these devices. Additionally uninsulated heat transfer surfaces of the bioreactor forces the heater to operate inefficiently, working harder to account for heat loss through uninsulated/non-heated surfaces.

Electrical heating jackets that can be wrapped about single walled bioreactor vessels are also known. These, however, are simple devices typically with a single temperature setting and heating rate and on/off functionality. They do not provide for adjustable or customized heat transfer and are not configured or adapted for use with different types of bioreactor vessels designed for different bioprocessing operations and having different temperature thresholds, different heat transfer properties and different rates at which the bioreactor vessel can be safely and efficiently heated. Such devices are also operated independent of any environmental condition, bioprocessing data or other information from the bioreactor. There is no feedback loop to provide even basic temperature limitations or potentially destructive applications of the heating jacket, such that use in connection with bioreactor vessels constructed from low melting point materials may result in overheating, damage to and/or fusing of the heating jacket and bioreactor. As such, these devices are not able to intelligently, efficiently or effectively transfer heat to bioreactors, particularly different types of bioreactors.

Furthermore, conventional heating jackets typically are constructed from opaque multilayered insulated fabric and/or rigid outer casings, purposefully designed for electrical and thermal insulation. They do not allow for direct visualization of the bioreactor culture through the heating jacket. Continuous, real time visual monitoring and regulation of the bioreactor culture, however, is a significant factor in managing bioprocessing, assessing ongoing biochemical reactions and ensuring high yield efficiency. Unobstructed transmission of light through a bioreactor vessel and heating apparatus is needed for accurate and clear, direct visual assessment of potential critical bioprocessing indicators, such as turbidity, coloration and tint. US 2016/184827 A1 for instance discloses a temperature control device for controlling the temperature of a container, wherein the temperature control device can be flexibly arranged on the wall of the container in a form-fitting manner. The temperature control device according to US 2016/184827 A1, however, does not allow for visualization of the bioreactor culture through the temperature control device.

In view of the above described deficiencies, there is a need to develop a heating apparatus that addresses deficiencies of the present bioreactor heating systems.

An object of the present disclosure is to provide a heating apparatus having a transparent body allowing for direct visualization of a bioreactor culture while the heating apparatus is coupled to a bioreactor and in operation. CN 108998376 A discloses a dynamic cell culture device and method based on a dielectric elastomer driver. The dynamic cell culture device according to CN 108998376 A discloses that a container body, a cover, a holder module and/or a heat generating sheet compartment can be made of a transparent material.

Another object of the present disclosure is to provide a heating apparatus that may be reusably coupled to various different types of bioreactors and allows for customized heat transfer specifically adapted to the needs of the attached bioreactor and bioreactor culture.

### SUMMARY

A heating apparatus for a bioreactor is provided, wherein the heating apparatus comprises: a flexible body that is transparent, having a transparent portion comprising a material with translucent properties, and detachably couplable to and configured to be positioned about and substantially enclose sidewalls defining a perimeter of a transparent bioreactor vessel for containing a bioreactor culture; a heating element coupled to the flexible body and arranged to transfer heat to the exterior sidewalls of the bioreactor vessel to uniformly heat the bioreactor culture, wherein the heating element is positioned throughout and spanning the extent of a heat transfer region of the flexible body, including the transparent portion, the heating element being configured as an electrically conductive, resistive wire, arranged in such a way that visibility through the transparent portion is not substantially obstructed, wherein the heating element is connectable to a controller for regulating heat transfer; and a sensor coupled to the flexible body to detect a temperature and/or a change in temperature of the heating apparatus and/or the bioreactor culture, wherein the sensor is connectable to the controller to monitor and/or regulate a temperature of the heating apparatus and/or bioreactor culture. Thus, direct visualization of a bioreactor culture while the heating apparatus is coupled to the bioreactor is achieved. It should be noted that CN 108998376 A does not disclose that the heat generating sheet itself can be made of a transparent material, only the container body, the cover, the holder module and/or a heat generating sheet compartment.

An embodiment relates to an aforementioned apparatus, wherein: the sensor and the heating element are configured as an integrated unit or component of the heating apparatus.

An embodiment relates to an aforementioned apparatus, wherein the heating element (comprises one or more electrically conductive wires forming a heat transfer portion of the flexible body, wherein the heat transfer portion of flexible body is transparent and the heating element is configured and arranged so as not to substantially obstruct visibility of the bioreactor culture through the heat transfer portion when the heating apparatus is coupled to the transparent bioreactor vessel.

An embodiment relates to an aforementioned apparatus, wherein the flexible body allows for visibility of the bioreactor culture through at least one or more opposing exterior sidewalls of the flexible body when positioned about and adjoining opposing exterior side walls of the transparent bioreactor vessel.

An embodiment relates to an aforementioned apparatus, wherein the heating apparatus is configured to be reusably and removably coupled to a first bioreactor and is configured to perform a first heating action adapted for the first bioreactor; wherein the heating apparatus is configured to be reusably and removably coupled to a second bioreactor and is configured to perform a second heating action adapted for the second bioreactor; and wherein the first heating action is different than the second heating action.

An embodiment relates to an aforementioned apparatus, wherein the first and second heating actions are selected from the group consisting of: heating to a predetermined temperature or temperature range; transferring heat at a predetermined temperature; transferring heat at a predetermined heating rate; heating or limiting applied heat based on a temperature threshold; heating or limiting applied heat based on a change in temperature; heating for a predetermined duration; and/or applying heat in accordance with a predetermined program.

An embodiment relates to an aforementioned apparatus, wherein the first and second heating actions are different in view of and are dependent upon one or more factors selected from the group consisting of: a type of bioreactor vessel coupled to the heating apparatus; a configuration and/or material property of a bioreactor vessel couplable to the heating apparatus; an intended operation of the couplable bioreactor vessel; and/or a desired temperature, temperature range or change in temperature of a bioreactor culture of the bioreactor vessel coupled to the heating apparatus.

An embodiment relates to an aforementioned apparatus, wherein the first and second heating action is transferring heat at a predetermined heating rate, wherein the first bioreactor is a single use bioreactor and the second bioreactor is a multi-use bioreactor, and wherein the heating apparatus is configured to heat the first transparent bioreactor vessel at a first predetermined rate and wherein the heating apparatus is configured to heat a second bioreactor vessel at a second predetermined rate higher than the first predetermined rate.

An embodiment relates to an aforementioned apparatus, wherein the flexible body is configured as a heating jacket and further comprises: a first flexible layer comprising a first transparent portion allowing for visibility through the first flexible layer; and a second flexible layer comprising a second transparent portion allowing for visibility through the second flexible layer, wherein the heating element is positioned between the first and second flexible layers, and wherein the bioreactor culture is viewable through the first and second transparent portions of the flexible body when the heating apparatus is coupled to the transparent bioreactor vessel.

Another aspect of the invention concerns a bioreactor system comprising: an aforementioned heating apparatus; and a controller for monitoring and/or regulating heat transfer, wherein the controller is connected to the heating element and the sensor.

An embodiment relates to an aforementioned bioreactor system, further comprising: a single use bioreactor comprising a plastic bioreactor vessel; and a multi-use bioreactor comprising a glass bioreactor vessel, wherein the heating apparatus is configured to be removably coupled to and operate in connection with the plastic bioreactor vessel and the glass bioreactor vessel and wherein the heating apparatus is configured to perform a first heating action to the plastic bioreactor vessel and perform a second heating action to the glass bioreactor vessel, wherein the first heating action is different from the second heating action.

An embodiment relates to an aforementioned bioreactor system, further comprising: a user interface operatively associated with the controller to allow a user to adjust a heating action of the heating apparatus.

An embodiment relates to an aforementioned bioreactor system, further comprising: a supplemental heater connected to the controller and coupled to the transparent bioreactor vessel for transferring heat to the transparent bioreactor vessel, wherein the controller is configured to coordinate and centrally regulate heat transfer through heating apparatus and supplemental heater.

An embodiment relates to an aforementioned bioreactor system, comprising: a first bioreactor for cultivating organisms, wherein the first bioreactor comprises a first transparent bioreactor vessel containing a first bioreactor culture; wherein the heating apparatus is operatively associated with the first transparent bioreactor vessel, wherein the heating apparatus is positioned about an exterior surface of the first bioreactor vessel or a bioreactor vessel component, wherein the heating apparatus transfers heat to the bioreactor culture, detects a temperature of the heating apparatus and/or the bioreactor culture, and is configured and arranged so as not to substantially obstruct visibility of the bioreactor culture; and wherein the controller is connected to the heating apparatus for regulating a temperature of the bioreactor culture and heat applied by the heating apparatus, wherein the heating apparatus is configured to perform a first heating action specific to and adapted for one or more parameters or conditions of the first bioreactor and is configured to perform a second heating action specific to and adapted for one or more parameters of a second bioreactor, wherein the first and second heating actions are different.

An embodiment relates to an aforementioned bioreactor system, wherein the system further comprises a supplemental heater coupled to and configured to transfer heat to the first transparent bioreactor vessel, wherein the controller is connected to the supplemental heater and is configured to centrally control and coordinate heat applied by the heating apparatus and the supplemental heater in order to regulate the temperature of the heating apparatus and/or the first bioreactor culture.

An embodiment relates to an aforementioned bioreactor system, further comprising the second bioreactor; wherein the first bioreactor is a single use bioreactor and the first transparent bioreactor vessel is a plastic transparent bioreactor vessel; and wherein the second bioreactor is a multi-use bioreactor comprising a glass bioreactor vessel; and wherein the heating apparatus is configured to be removably coupled to and operate in connection with the first and second transparent bioreactor vessels.

An embodiment relates to an aforementioned bioreactor system, wherein the heating apparatus comprises: a flexible body comprising one or more transparent portions allowing for visibility of the first bioreactor culture when the heating apparatus is coupled to the portion of the bioreactor; a heating element configured and arranged so as not to substantially obstruct visibility of the bioreactor culture through the one or more transparent portions of the flexible body when the heating apparatus is coupled to the portion of the first bioreactor; and a temperature sensor connected to the controller, wherein the temperature sensor detects a temperature or change in temperature of the flexible body and/or the first bioreactor culture.

An embodiment relates to an aforementioned bioreactor system, wherein the heating apparatus is integrally formed with, coupled to, attached to or connected to a component of the bioreactor selected from the group consisting of: a transparent bioreactor vessel wall; an impeller of the bioreactor; an impeller shaft of the bioreactor; a diptube of the bioreactor; and/or a spin filter of the bioreactor.

Another aspect of the invention concerns a bioreactor temperature regulating method comprising the steps of: operatively associating an aforementioned heating apparatus with a bioreactor vessel; and applying heat to the bioreactor vessel or a bioreactor culture contained within the bioreactor vessel using the heating apparatus.

An embodiment relates to an aforementioned method, further comprising: detecting a condition, parameter or change thereof of the bioreactor culture and/or the heating apparatus using the heating apparatus; and regulating a temperature of the bioreactor culture and/or heating apparatus based on the detected condition, parameter or change thereof of the bioreactor culture and/or the heating apparatus.

An embodiment relates to an aforementioned method, further comprising the steps of: operatively associating a heating apparatus with a first bioreactor vessel, wherein the heating apparatus has a transparent body; performing a first heating action adapted for the first bioreactor vessel or a first bioreactor culture contained therein using the heating apparatus; operatively associating a heating apparatus with a second bioreactor vessel; performing a second heating action adapted for the second bioreactor vessel or a second bioreactor culture contained therein using the heating apparatus, wherein the first and second heating actions are different; and directly visualizing the first bioreactor cultures through the transparent body when heating apparatus is operatively associated with the first bioreactor and directly visualizing the second bioreactor culture through the transparent body when heating apparatus is operatively associated with the second bioreactor.

### BRIEF DESCRIPTION OF THE DRAWINGS

The following drawings illustrate exemplary embodiments and are not intended to limit the scope of the claims. Similar elements may be referred to by common or like reference numerals throughout the figures to facilitate description. These drawings are not necessarily drawn to scale.

FIG. 1A illustrates a first exemplary heating apparatus for a bioreactor.

FIG. 1B is an exploded view of the heating apparatus of FIG. 1A.

FIG. 2 illustrates a second embodiment of a bioreactor heating apparatus.

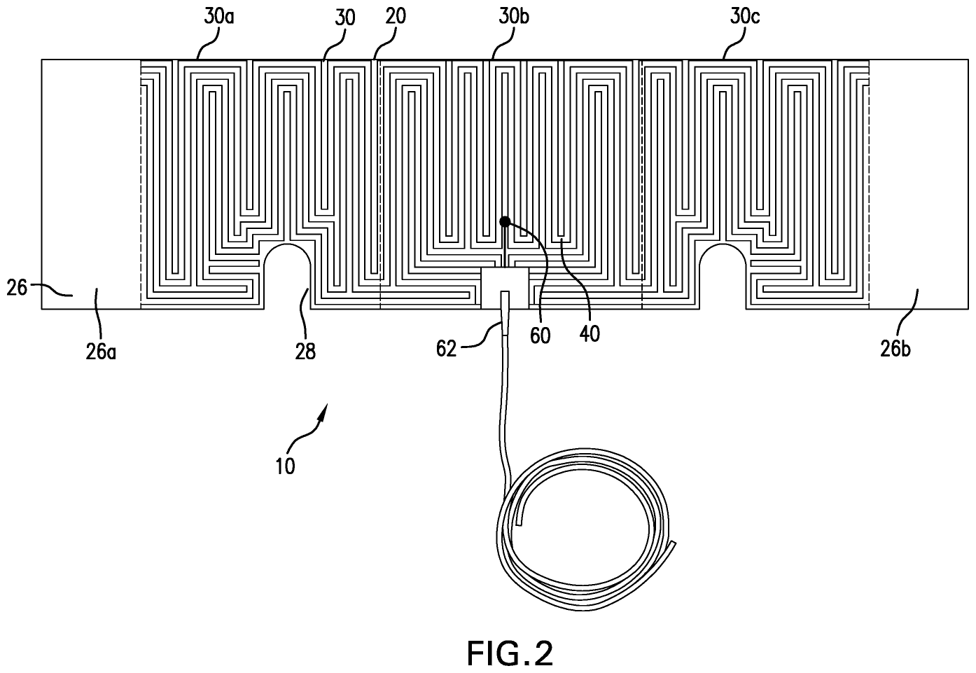

FIG. 3 shows the exemplary heating apparatus of FIG. 1A positioned about a bioreactor.

FIG. 4 shows an exemplary bioreactor system including the exemplary heating apparatus of FIG. 1A.

FIG. 5 is a schematic diagram illustrating a third embodiment of a bioreactor heating apparatus.

FIG. 6 illustrates a fourth embodiment of a bioreactor heating apparatus.

FIG. 7 shows a first exemplary method for regulating a temperature of a heating apparatus and/or bioreactor culture using a heating apparatus.

FIG. 8 shows a second exemplary method for using a heating apparatus.

### DETAILED DESCRIPTION

For illustrative purposes, the general principles of the present disclosure are described by referencing various exemplary embodiments. Although certain embodiments of the disclosure are specifically described herein, one of ordinary skill in the art will readily appreciate that the same principles may be equally applicable to, and can be employed in other variations and embodiments. The present disclosure is not limited in scope or application to any particular described embodiments. Additionally, the terminology used herein is for the purpose of description and illustration, not limitation.

Various embodiments are described hereinafter with reference to the figures. These figures are only intended to facilitate the description of the exemplary embodiments. They are not an exhaustive description nor do they limit the scope of the disclosure. An illustrated embodiment does not necessarily embody all the aspects, features or advantages of the subject matter of the present disclosure. Additionally, any aspect, feature or advantage described in conjunction with a particular embodiment is not necessarily limited to that embodiment and may be embodied or practiced in other embodiments, irrespective of whether illustrated or described herein.

For purposes of the present Detailed Description, the singular forms “a”, “an”, and “the” may include plural references unless the context clearly dictates otherwise. Thus, for example, reference to “a heating element” may include a plurality of heating elements and equivalents thereof known to those skilled in the art. The terms “a” (or “an”), “one or more” and “at least one” may be used interchangeably herein. Additionally, the terms “comprising”, “including”, “composed of” and “having” may be used interchangeably.

As used herein, “bioreactor culture” refers to any content of a bioreactor vessel or bioreactor, such as but not limited to cultures of organisms, cells, tissues, other biological and/or chemical components, etc.

As used herein, “transparent” refers to a range of light transmission that includes complete transparency to substantial transparency or some translucent properties, wherein a majority of light propagates through a material, allowing for visibility without substantial obstruction of the field of view. An operator of the bioreactor should essentially be able to see the color, content level, agitation, homogeneity, et cetera, of the interior of the bioreactor, i.e. the operator should be able to visually determine the state of the bioreaction through the heating apparatus (as the operator could with a bioreactor without such a heating apparatus). This may be achieved e.g. by configuring the electrically conductive, resistive (heating) wire to not lower the light transmissivity of the heating apparatus by more than 10%, preferably not more than 20%, more preferably not more than 30%, most preferably not more than 50%. The electrically conductive, resistive wire may e.g. be configured to take up no more than 1%, preferably no more than 5%, more preferably no more than 10%, even more preferably no more than 20%, even more preferably no more than 30%, most preferably no more than 50% of the surface area of the heat transfer region.

As used herein, “sensor” refers to any device, element or item that detects a parameter, condition, property or change thereof and “detect” or “detection” refers to any direct or indirect means for measuring, sensing, calculating or determining. For example, a temperature sensor may include both a device that directly senses or measures temperature as well as a device that indirectly determines temperature or a change in temperature based on another parameter.

### Heating Apparatus

The present disclosure is directed to a heating apparatus 10 for a bioreactor 70, a bioreactor system 80 and a method for heating and/or regulating a temperature of one or more bioreactors 70 and/or the heating apparatus 10. In an exemplary embodiment, the heating apparatus 10 and bioreactor system 80 is configured for the safe, efficient, customized and intelligent temperature regulation of the heating apparatus 10 and/or one or more bioreactors 70 as well as configured for clear visualization of a bioreactor culture through a transparent portion 30 of heating apparatus 10 to facilitate bioprocessing, cultivation and assessment of the bioreactor culture. The heating apparatus 10 may be configured for use in connection with different types of bioreactors 70, including single-use bioreactors as well as continuous use, multi-use or batch-fed bioreactors, wherein various heat transfer parameters are customized for the corresponding bioreactor 70 and/or bioreactor culture, such the rate of heating, set temperature, temperature thresholds, changes in temperature, heating duration, heating cycle, etc. An exemplary method for heating and/or regulating a temperature of the heating apparatus 10 and/or bioreactor 70 may involve using a heating apparatus 10 to transfer heat to a bioreactor vessel 72; determining a temperature or change in temperature of the heating apparatus 10 and/or bioreactor contents; regulating heat transfer based on the determined temperature and/or change in temperature and monitoring the bioreactor culture and bioprocessing by direct visualization of the bioreactor culture through a transparent portion 30 of heating apparatus 10.

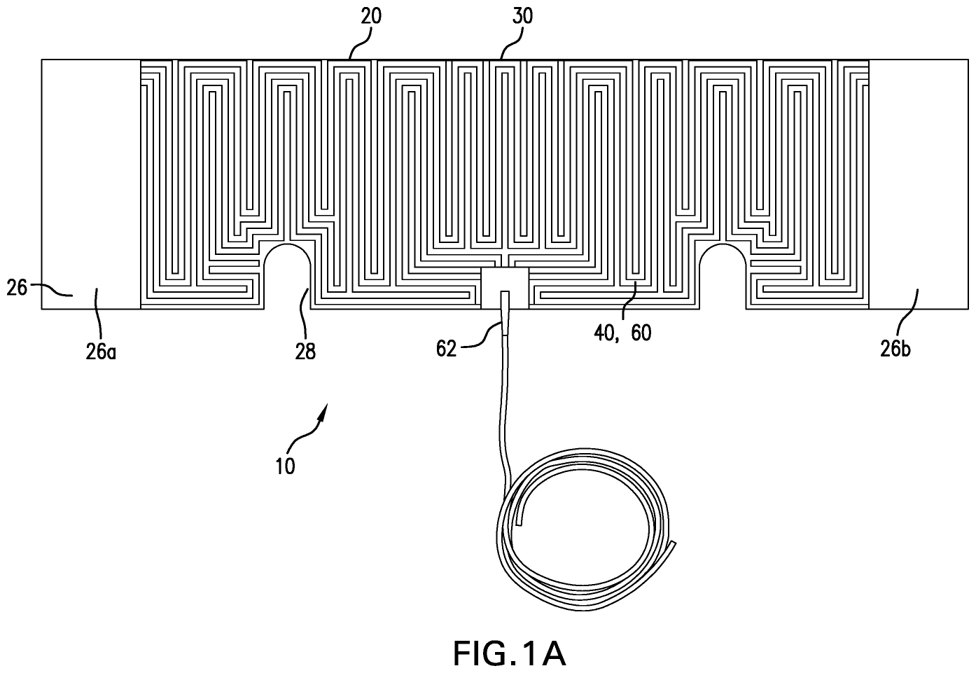

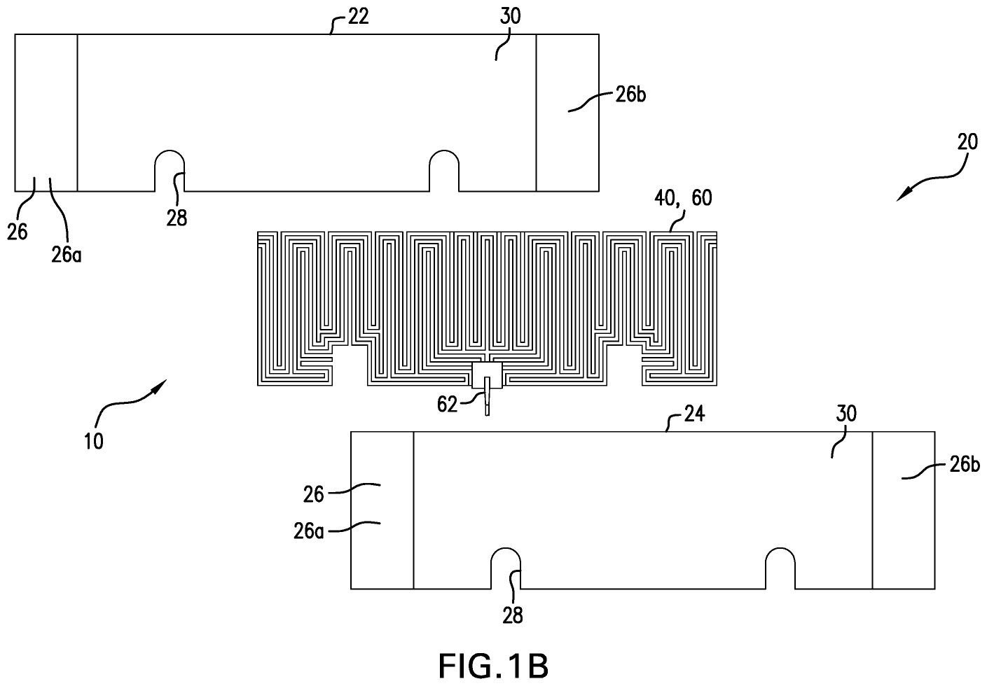

FIGS. 1A-1B and 3-4 illustrate an exemplary embodiment of a heating apparatus 10 for one or more bioreactors 70, wherein heating apparatus 10 has a flexible body 20 detachably coupled to a bioreactor vessel 72 and having one or more transparent portions 30. Heating apparatus 10 further includes one or more heating elements 40 for transferring heat to bioreactor 70 and one or more sensors 60 for determining a condition or parameter of heating apparatus 10 and/or the contents of a bioreactor vessel 72. In an exemplary embodiment, heating element 40 and sensor 60 may a single, integral unit or component of heating apparatus 10, functioning to both transfer heat and detect temperature. A controller 50 connected to or otherwise operatively associated with heating element 40 and/or sensor 60 regulates the temperature of a bioreactor culture contained within bioreactor vessel 72 and/or heating apparatus 10. In one embodiment, controller 50 may be a part, a component of, included in or otherwise operatively associated with heating apparatus 10 or a heating apparatus system. In another embodiment, controller 50 may be a separate device, such as a biocontroller of a bioreactor system 80. In an exemplary embodiment, heating apparatus 10 may be reusably and reattachably coupled to and is adapted for use with various different types of bioreactors 70 requiring different heat transfer functionality.

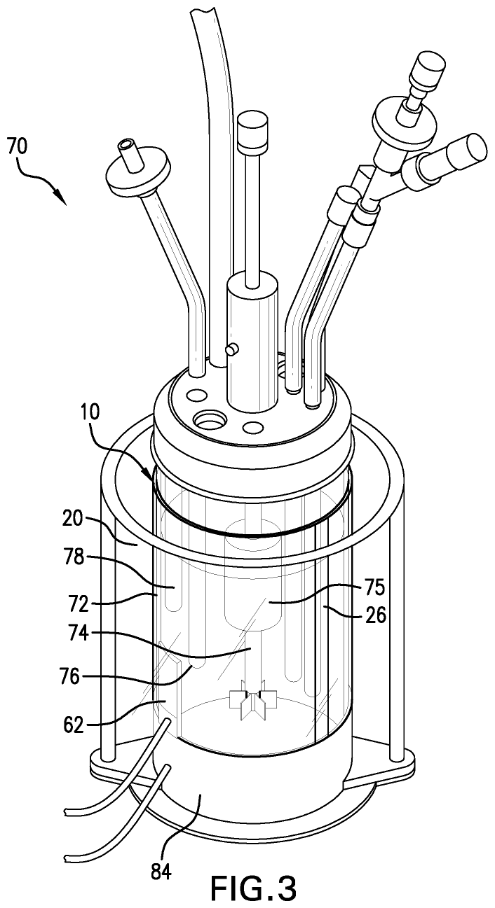

In the exemplary embodiment of FIGS. 1A-1B and 3, flexible body 20 of heating apparatus 10 is configured to be adjustably positioned about and placed in direct contact with the exterior surface of a transparent, cylindrical bioreactor vessel 72, so as to fully encircle the circumference and enclose the sidewalls of bioreactor vessel 72 for efficient and uniform heat transfer. Flexible body 20 may be configured as a wrap, jacket, blanket or other configuration adapted to substantially or fully conform to, enclose and insulate the perimeter and exterior sidewalls of bioreactor 70. In one embodiment, a substantial portion, the majority or the entirety of the sidewalls or surfaces of bioreactor vessel 72 are encased by heating apparatus 10. The shape of flexible body 20 preferably corresponds and facilitates secure attachment to bioreactor vessel 72. For example, flexible body 20 and heating apparatus 10 may have a generally rectangular configuration conforming to and adapted to surround the exterior sidewalls of a cylindrical bioreactor or may have a trapezoidal or asymmetrical configuration to accommodate conical or other variable diameter bioreactors. In one embodiment, flexible body 20 may have a prefabricated 3-dimensional structure, such as a cylindrical, conical or curved configuration, to facilitate attachment to a bioreactor vessel 72, In the embodiment illustrated in FIGS. 1A-1B and 3, flexible body 20 can be arranged and configured in multiple positions, e.g., a flattened configuration facilitating for storage (FIG. 1A), a cylindrical configuration fully encircling the circumference of the exterior sidewall of a cylindrical bioreactor vessel 72 (FIG. 3) or other shape and/or arrangement conforming to the exterior surface of bioreactor vessel 72. Flexible body 20 may also include one or more cut outs 28 along its length to facilitate positioning of heating apparatus 10 around bioreactor power cords, connectors, sampling ports or other components protruding from a surface of bioreactor vessel 72. Heating apparatus 10 and flexible body 20 may be configured to fit bioreactors vessels 72 of different sizes, including small laboratory benchtop bioreactors less than 1000 mL or ranging from 250 mL to 1000 mL in capacity. In other embodiments, heating apparatus 10 and flexible body 20 may be sized to fit bioreactors that are 1 L, 2 L, 3 L or larger in size.

As shown in FIGS. 1A-1B, flexible body 20 may include one or more fasteners or fastener sections 26 to adjustably position and secure flexible body 20 to bioreactor 80. Fastener 26 is preferably designed to allow heating apparatus 10 and flexible body 20 to be detachably and reattachably coupled to bioreactor 80 permitting reuse. In one embodiment, fastener or fastener sections 26a, 26b may be positioned on or near opposing ends of flexible body 20 (FIG. 1A), allowing a user to wrap and secure flexible body 20 about bioreactor vessel 72 by overlapping and attaching a first fastener section 26a to second fastener 26b (FIG. 3). Fasteners 26 may be configured as hook and loop fasteners, reusable adhesives, hooks, clips, clamps, snaps or other types of fasteners.

Flexible body 20 may include one or more transparent portions 30 that allow for clear visibility of the bioreactor culture within a transparent bioreactor vessel 72 when surrounded by heating apparatus 10. Such direct visualization of the bioreactor culture facilities bioprocessing analysis, assessment, monitoring and cultivation of the bioreactor culture. In one embodiment, flexible body 20 is entirely transparent, including transparent fastener sections 26, allowing for visibility through all surfaces of heating apparatus 10. In an alternative embodiment, flexible body 20 may have one or more opaque portions, such as an opaque fastener section 26, as well as one or more transparent portions 30a, 30b, 30c, positioned or arranged to allow for visibility through a majority or a substantial portion of heating apparatus 10. Preferably, transparent portions 30 are positioned and arranged such that when heating apparatus 10 is attached to bioreactor vessel 72, the bioreactor culture may be clearly viewed through one or more pairs of opposing exterior sidewalls, surfaces or portions of flexible body 20 adjacent to opposing exterior sidewalls of transparent bioreactor vessel 72, such that an individual has a direct field of view through opposing exterior sidewalls of a transparent bioreactor vessel 72 and through opposing exterior sidewalls of heating apparatus 10 enclosing bioreactor vessel 72. Unobstructed transmission of light and clear visualization of the bioreactor culture through opposing transparent exterior sidewalls of bioreactor vessel 72 and heating apparatus 10 improves visual assessment of the bioreactor culture, enabling analysis that may not be otherwise easily discerned. For example, accurate assessment of turbidity, coloration and/or tint to gauge the progress and/or status of a bioreactor culture is preferably assessed when there is a direct path/transmission of light through bioreactor vessel 72 and heating apparatus 10. In another embodiment, transparent portions 30 are arranged to allow for visibility through multiple adjoining and/or opposing exterior sides or faces of flexible body 20. In another embodiment, transparent portions 30 comprise at least 50% or at least 75% of flexible body 20. In another embodiment, when heating apparatus 10 is attached to and substantially or fully encloses the sidewalls of transparent bioreactor vessel 72, at least 50% of the sidewalls or at least 75% of transparent bioreactor vessel 72 enclosed by heating apparatus 10 allows for direct visualization of the bioreactor culture.

In the embodiment shown in FIG. 1B, flexible body 20 may have a multilayer material construction including a first layer 22, 24 having one or more transparent portions 30 and a second layer 24 including one or more corresponding transparent portions 30. First and second layers 22 may be configured as a sheet made from a polymeric material, preferably a heat resistant polymeric material such as silicone. In another embodiment, layers 22, 24 may be made from a transparent metal. Transparent portions 30 of first and second layers 22, 24 similarly may be made from a transparent polymeric material, preferably a heat resistant polymeric material such as silicone, or a transparent metal. In one embodiment, the first and second layers 22, 24 define a substantial extent or the entirety of flexible body 20 and is constructed from a transparent polymeric material allowing for visualization through the entirety of the first and second layers 22, 24 forming flexible body 20. In another embodiment, flexible body 20 is further defined by one or more fastener sections 26, which may be attached to and adjoin first and second layers 22, 24. The one or more fastener sections 26 may be constructed from the same or similar transparent polymeric materials as first and second layers 22, 24, permitting full visibility through the entirety of flexible body 20. Alternatively, one or more fastener sections 26 may be opaque but preferably only forms a small portion of flexible body 20.

Heating apparatus 10 and flexible body 20 further includes one or more heating elements 40 that may be disposed on, embedded in, attached to, coupled to or otherwise operatively associated with flexible body 20. Heating element 40 is configured and adapted to transfer heat through flexible body 20 to a bioreactor vessel 72 adjacent, adjoining or otherwise coupled to heating apparatus 10. Specifically, heating element 40 is configured and adapted to transfer heat through the sidewalls of bioreactor vessel 72 to a bioreactor culture. In one embodiment, heating element 40 may be configured, arranged and adapted to uniformly apply heat to the sidewalls of bioreactor vessel 72. Heating element 40 may be configured and adapted to transfer heat to different types of bioreactors 70, including without limitation single use bioreactors having bioreactor vessels 72 constructed from clear plastic materials or multi-use or continuous use bioreactors with bioreactor vessels 72 constructed from glass. Heating apparatus 10 may further be configured and programmed to heat to each bioreactor 70 differently, e.g., different applied heating rates, different temperature thresholds and/or set points, different heating durations, different heating profiles, cycles or programs, etc., to provide customized and safe heating of bioreactor vessel 72, the contents therein and heating apparatus 10.

In one embodiment, heating elements 40 may be directly disposed on, embedded in, attached to, coupled to or otherwise operatively associated with any portion of first and/or second layers 22, 24, including transparent portion 30 and/or attachment portions 26. In another embodiment, heating element 40 may be disposed on, embedded in, attached to, coupled to or otherwise operatively associated with a third layer having the same or similar construction and material composition and properties as that of first and second layers 22, 24. In one embodiment heating elements 40 and/or a third layer containing heating elements 40 is preferably positioned between and insulated by first and second layers 22, 24.

Heating elements 40 may be configured and constructed as any type of element enabling heat transfer and does not substantially impede the transparency or obstruct visualization through flexible body 20, specifically through one or more transparent portions 30. In the embodiment of FIGS. 1A-1B, heating element 40 is configured as electrically conductive, resistive wire that releases heat when an electrical current is passed through the wire. In one embodiment, the wire may be prefabricated and disposed on, positioned on, embedded in, coupled to, adhered to, molded together with or otherwise coupled to a portion of flexible body 20. In another embodiment, the wire may be printed on, etched into, or otherwise formed and attached to flexible body 20. The wire may be configured as a thin or fine wire such that even if densely arranged on or within a portion of transparent portion 30, visibility through such transparent region is not substantially obstructed and an individual is still able to clearly view the bioreactor culture through transparent portion 30. In one embodiment, the wire may be made from copper, aluminum, nickel, platinum, alloys thereof, a transparent metal or like conductive materials. The wire may be preformed and coupled to flexible body 20 or a conductive heat transfer circuit may be formed by cutting, etching, deposition or other methods. The wire may e.g. have a circular cross-section, but a square, triangular or other cross-section is also conceivable. Preferably, the one or more heating elements 40 are arranged in association with flexible body 20 so as to enable substantially uniform heating throughout a heat transfer region of flexible body 20. In one embodiment, the heat transfer region of flexible body 20 includes transparent portions 30. In the embodiment shown in FIG. 1A, heating element 40 is densely arranged in a series of closely positioned rows, columns and/or convolutions throughout a heat transfer portion of flexible body 20. One or more heating elements 40 are densely positioned throughout and span the extent of a heat transfer region of flexible body 20, including transparent portion 30, but as illustrated in FIG. 3, the heat transfer region of flexible body 20 and transparent portion 30 remains fully transparent or substantially transparent.

Heating apparatus 10 and flexible body 20 further includes one or more sensor 60 disposed or positioned on, embedded in, attached or coupled to or otherwise operatively associated with flexible body 20 to measure, sense, detect and/or determine one or more parameters or conditions of heating apparatus 10 and/or a coupled bioreactor 70. Sensors 60 may be any type of sensor that facilitates operation of heating apparatus 10, regulation of heat transfer regulation and/or bioprocessing. In one embodiment, sensors 60 may be a temperature sensor, capacitive sensor, photoelectric sensor, ultrasonic sensor and/or other sensor. In an exemplary embodiment, a sensor 60 may be used to directly or indirectly determine a temperature of flexible body 20 and/or a bioreactor culture and may be configured as a thermocouple, thermistor or resistance temperature detector. Multiple sensors 60 may be coupled to and positioned throughout flexible body 20. Preferably, multiple integrated temperature sensors may be arranged throughout flexible body 20. As shown in the exemplary embodiment of FIGS. 1A-1B, heating element 40 and a temperature sensor 60 may be configured as a single, integrated component or unit. For example, heating element 40 may be a conductive, resistive wire configured to transfer heat and determine the temperature of flexible body 20 and/or the bioreactor culture based on a measured voltage and/or determined change in resistance of the wire. In another embodiment, in addition to or as an alternative to a temperature sensor, heating apparatus 10 may include a sensor 60 that may detect a material property of bioreactor vessel 72, size and/or configuration of bioreactor vessel 72 or potentially a cell concentration or density of the bioreactor culture.

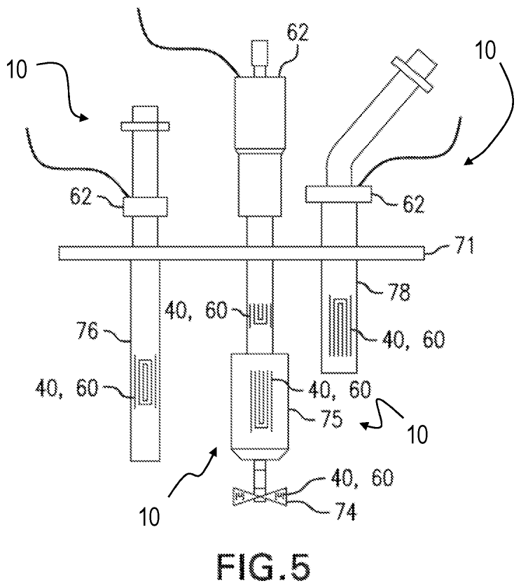

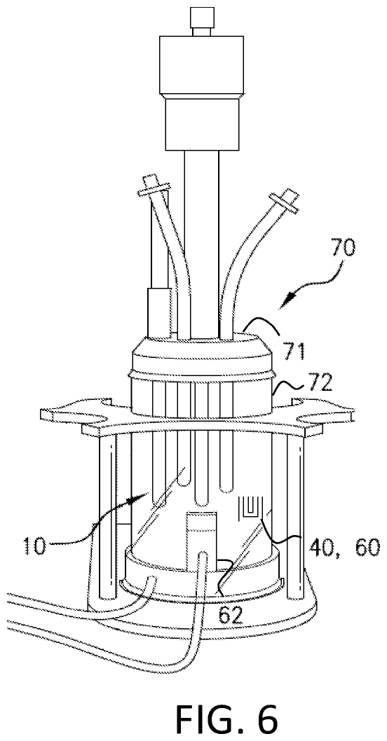

In alternative exemplary embodiments, as illustrated in FIGS. 5-6, heating apparatus 10 may be integrated with a component of bioreactor 70 and/or bioreactor vessel 72. In one embodiment, heating apparatus 10 may have a body integrally formed with, attached to or coupled to bioreactor 70 and/or bioreactor vessel 72. In one embodiment, the body of heating apparatus 10 may be constructed from a biocompatible material and configured as a flexible body 20, such as a silicone film or layered construct, which is coupled to, attached to or otherwise operatively associated with one or more heating elements 40 and one or more sensors 60. For example, heating elements 40 and sensor 60 may be embedded in and otherwise insulated by flexible body 20. In another embodiment, heating elements 40 and sensors 60 are not supported by a carrier or body and instead are integrally formed with, attached to or coupled to bioreactor 70 and/or bioreactor vessel 72. In the embodiment shown in FIG. 5, heating apparatus 10 and heating elements 40 and sensors 60 may be attached to, adhered to, integral with or otherwise operatively associated with one or more portions of impeller 72, including without limitation an impeller shaft and/or impeller blade, a spin filter 75, a dip tube 76 or bioreactor culture sensor 78. In the embodiment shown in FIG. 6, Heating apparatus 10 and heating elements 40 and sensors 60 may be attached to, adhered to, integral with or otherwise operatively associated with a transparent sidewall of bioreactor vessel 72 (FIG. 6). Such arrangement allows a user to clearly view the contents of bioreactor 72 while heating apparatus 10 is in operation. In an exemplary embodiment, these bioreactor components may be substantially or fully transparent and made from transparent materials, such as glass or clear plastics, and heating apparatus 10 and heating apparatus body may be substantially or fully transparent. This further facilities visibility of the contents within the bioreactor vessel, allowing an individual to view the bioreactor culture through one or more bioreactor components and heating apparatus 10.

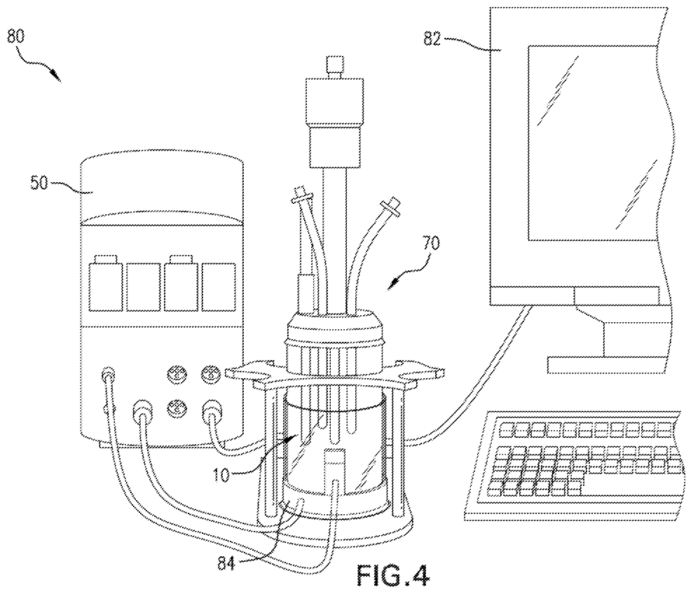

Connector 62 of heating apparatus 10 connects heating element 40 and/or sensor 60 to a controller 50 and/or power source for operating heating apparatus 10. Heating element 40 and sensor 60 are connected to and/or otherwise operatively associated with a controller 50 for regulating heat transfer and/or a temperature of heating apparatus 10 and/or the contents of bioreactor vessel 72. In one embodiment, controller 50 may be part of, integral with and/or otherwise operatively associated with heating apparatus 10. For example, controller 50 may be an integral component of connector 62, which also functions to connect heating element 40 and/or sensor 60 with a power source. In the exemplary embodiment shown in FIG. 4, heating element 40 and sensor 60 are connected via connector 62 to a controller 50, configured as a biocontroller that is part of a bioreactor system 80.

Controller 50 may be used to monitor the temperature and/or change in temperature of heating apparatus 10 and/or the contents of bioreactor vessel 72 as well as heat transfer there between. In an exemplary embodiment, controller 50 and heating apparatus 10 are configured, programmed and adapted to provide customized heat transfer, preferably specific to and targeting the unique requirements and/or limitations of different types of bioreactors 70, different bioreactor cultures and different bioprocessing applications. For example, desired and tolerable heating activity, heat transfer action or heat transfer functions for a single use bioreactor 70 and a corresponding single use clear plastic bioreactor vessel 72 requiring a lower rate of heat transfer, having a lower maximum temperature threshold/lower melting point and having a shorter durational tolerance for heating may be different than a multi-use or continuous use bioreactor 70 including a glass bioreactor vessel 72 that requires a higher rate of heat transfer, a higher maximum temperature threshold/higher melting point and longer durational tolerance for heating. Customized heating and heat transfer using heating apparatus 10 therefore enables efficiently and safe operation of heating apparatus 10 as well as effective heating for the attached bioreactor 70. In one embodiment, heating apparatus 10 and controller 50 are configured and programmed to adjust heating and/or temperature regulation based on various different factors, including but not limited to the type of bioreactor 70 coupled to heating apparatus 10, a configuration and/or material property of a bioreactor vessel 72 coupled to the heating apparatus 10, a material property of heating apparatus 10 and/or flexible body 20, an intended bioprocessing operation of the bioreactor 70, an intended yield or bioprocessing outcome, a desired temperature or change in temperature of a bioreactor culture, a desired temperature or change in temperature of heating apparatus 10, a detected temperature or change in temperature of the heating apparatus 10 or bioreactor culture, detected heat introduced by bioreactor system components and actuators (such as from the agitator motor of impeller 74), a detected turbidity or clarity of a bioreactor culture, a detected color or tint of a bioreactor culture, a detected cell concentration or density of a bioreactor culture or user input/user designated parameters. In any given situation, controller 50 and heating apparatus 10 may take the above factors into considerations when determining a heating activity, heat transfer action or heat transfer function, such as an applied heating rate; heating temperature; heating duration; heating cycles, profile, or programs; initial, final and/or interim temperature set points, etc., to provide customized, efficient, high yield and safe heating for different bioreactors 70, different bioreactor vessels 72, different bioreactor cultures and/or different bioprocessing applications.

One or more sensors 60 operatively associated with controller 50 provides a feedback loop to facilitate continuous, real time monitoring and assessment of the bioreactor culture and condition of heating apparatus 10. Adjustments to the heat transfer functionality of heating apparatus 10 based on data from one or more sensors 60 enables customized and efficient bioprocessing.

In an exemplary embodiment, heating apparatus 10 and controller 50 are preprogramed so as to require nominal setup and/or oversight by a user when using heating apparatus 10. Heating apparatus 10, controller 50 and/or bioreactor system 80 may be configured as smart devices and systems, wherein heating apparatus 10 may automatically detect and determine an appropriate heating activity or function upon connection with controller 50 and/or coupling to bioreactor 70 based on: pre-programmed heat transfer functions and settings for different applications and bioreactor equipment received from controller 50; environmental or physical parameters or conditions detected by sensors 60 relating to the bioreactor 70, bioreactor vessel 72 and/or heating apparatus 10; and/or information inputted/directed by a user. In one embodiment, upon coupling heating apparatus 10 to a bioreactor vessel 72, bioreactor 70 and/or controller 50, heating apparatus 10 automatically detects and implements an initial heating function specifically adapted for the corresponding bioreactor 70, bioreactor vessel 72, bioreactor culture and/or intended bioprocessing application and/or restricts heating functionality that would be detrimental and/or have a negative impact on heating apparatus 10, bioreactor 70, bioreactor vessel 72 and/or the bioreactor culture. For example, heating apparatus 10 and controller 50 may be pre-programmed with heat transfer functions and actions adapted for different types of bioreactors, different types of bioreactor vessels, different bioreactor cultures and/or intended bioprocessing applications. Upon connecting heating apparatus 10 to bioreactor 70 and bioreactor vessel 72, sensor 60 may function to determine which of the pre-programmed options is most suitable for the intended use. Similarly, upon receiving user input from controller 50 as to an intended bioprocess application, type of bioreactor culture being cultivated or other information, the pre-programed heating actions and heat transfer functions may be further sorted and filtered to determine a recommended course of action for heating using heating apparatus 10.

### Bioreactor System

The heating apparatus 10 of the present disclosure may be part of a novel bioreactor system 80. In one embodiment, bioreactor system 80 may include any of the described embodiments of heating apparatus 10, a controller 50 and one or more bioreactors 70. In one embodiment, bioreactor system 80 may include two or more different types of bioreactors 70, such as a single use bioreactor, multi-use bioreactor or continuous use bioreactor. Additionally, bioreactor system 80 may include two bioreactor vessels 72 constructed from different materials, such as glass and clear plastic. In one embodiment, bioreactor vessels 72 may be transparent. In another embodiment, bioreactor vessel (72 are opaque but may change colors based on the pH value of a bioreactor culture contained therein.

In the embodiment shown in FIG. 4, bioreactor system 80 may include a heating apparatus 10, controller 50, one or more bioreactors 70 and a display and/or user interface 82. Optionally, bioreactor system 80 may further includes a base heater 84 that may be connected to controller 50, allowing controller 50 to centrally regulate the temperature of a bioreactor culture of one or more bioreactor vessels 72 by controlling heat transfer through both heating apparatus 10 and base heater 82.

Heating apparatus 10 and bioreactor system 80 offers several advantages and benefits, namely providing improved monitoring and customized, adjustable, efficient, effective and safe temperature regulation. In particular, heating apparatus 10 is reusable and is adapted to be used in connection with various different bioreactors 70, bioreactor vessels 72, bioreactor systems 80 and bioreactor cultures, obviating the need to have multiple customized heating apparatuses for specific bioreactors, bioreactor systems or intended bioprocessing applications. Heating apparatus 10 is further configured to perform heat transfer functions specifically adapted for the coupled bioreactor. Sensors 60 further provides a continuous feedback loop of conditions and parameters of heating apparatus 10 and/or a bioreactor culture to facilitate continuous real time monitoring, assessment and adjustment of bioprocessing conditions and parameters, including but not limited to adjustment of heat transfer from heating apparatus 10. The transparent portions 30 of heating apparatus 10 enable immediate and clear visibility of the bioreactor culture, further assisting with monitoring, assessment and adjustment of bioprocessing conditions and parameters as needed.

### Method For Heating A Bioreactor

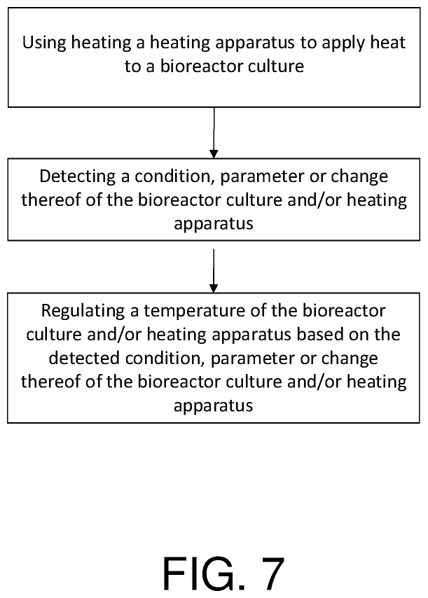

The present disclosure is further directed to a method for heating a bioreactor 70 using heating apparatus 10. In the exemplary embodiment illustrated in FIG. 7, the method involves: using any of the described heating apparatus 10 to apply heat to a bioreactor vessel 72 operatively associated with a heating apparatus 10; determining a condition, parameter or change thereof of the bioreactor culture and/or heating apparatus 10; and regulating heat transfer using heating apparatus 10 based on the determined condition, parameter or change thereof of the bioreactor culture and/or heating apparatus 10. Heating apparatus 10 may be operatively associated with bioreactor 70 using any of the methods in the present disclosure, such as removably and reusably attaching heating apparatus 10 to encase an exterior sidewall of bioreactor vessel 72. In one embodiment, the method further involves the step of removing heating apparatus 10 from a first bioreactor vessel 72, such as a single-use plastic bioreactor vessel, and reusably attaching heating apparatus 10 to a different second bioreactor vessel, such as a multi-use or continuous use glass bioreactor vessel.

In other embodiments, heating apparatus 10 may be integrally formed with a component of bioreactor 70, such as a transparent sidewall of bioreactor vessel 72, an impeller 74, an impeller shaft, an impeller blade, a spin filter 75, a dip tube 76 or a bioreactor sensor 78 (FIGS. 5-6). The heating apparatus 10 and integrally connected bioreactor component may be detachably coupled to a first bioreactor70 or bioreactor vessel 72, such as a single-use transparent plastic bioreactor vessel, and subsequently detached and reusably coupled to a different second bioreactor 70 or bioreactor vessel 72, such as a multi-use or continuous use glass bioreactor vessel. For example, the integral heating apparatus 10 and bioreactor component maybe inserted through and/or reattachably connected to a bioreactor lid 71 of a first single use transparent plastic bioreactor vessel 72 and may be subsequently removed and reusably inserted through and connected to a second lid 71 of a second multi-use or continuous use glass bioreactor vessel 72.

Heating apparatus 10 may be used to perform a heating function to the first bioreactor 70 and/or bioreactor vessel 72 that is different than a heating function performed with respect to the second bioreactor 70 and/or bioreactor vessel 72. The performed heating action may be customized or specifically adapted for the correspondingly attached bioreactor 70 and/or bioreactor vessel 72. In one embodiment, the method involves regulating applied heat from heating apparatus 10 and/or a temperature of the bioreactor culture and/or heating apparatus 10 based on various detected conditions, parameters or changes thereof of a heating apparatus 10 and/or a bioreactor culture contained within bioreactor 72, as previously described above.

In one embodiment, the method further involves the step of directly visualizing the bioreactor culture through a transparent portion 30 of heating apparatus 10. For example, when heating apparatus 10 is configured as a heating wrap or jacket positioned around and enclosing transparent bioreactor vessel 72, an individual is able to directly view a bioreactor culture through the sidewalls of a transparent bioreactor vessel 72 and the transparent sidewalls of heating apparatus 10 encircling a circumference of bioreactor vessel 72. In another embodiment, bioreactor 70 and/or a component of bioreactor vessel 72, such as an impeller 72 or spin filter 75, may be transparent and integrated with a transparent heating apparatus 10 integrated therewith, allowing an individual to view a bioreactor culture through the transparent component of bioreactor 70 and bioreactor vessel 72 as well as the transparent heating apparatus 10.

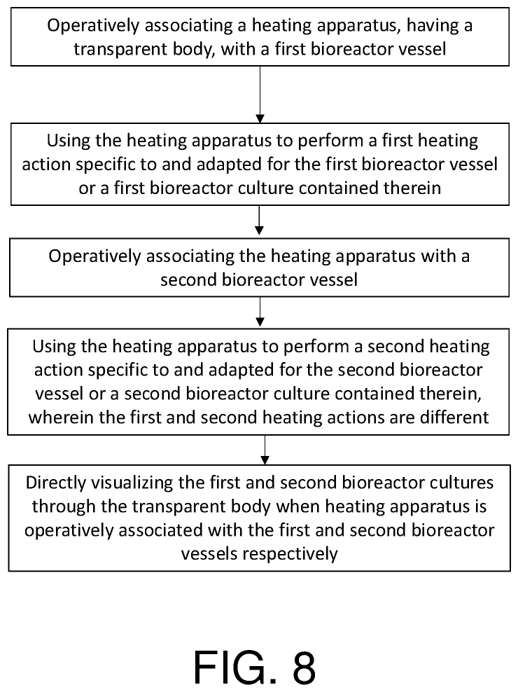

As illustrated in FIG. 8, another exemplary method may involve the steps of: operatively associating a heating apparatus 10 having a transparent body to a first bioreactor vessel 72; using heating apparatus 10 to perform a first heating activity specific or adapted for the first bioreactor vessel 72 or second bioreactor culture; operatively associating the heating apparatus 10 to a second bioreactor vessel 72; using heating apparatus 10 to perform a second heating activity specific or adapted for the second bioreactor vessel 72 or second bioreactor culture, wherein the first and second heating actions are different; and directly visualizing the contents of the first and second bioreactor vessel 72 through the transparent body of heating apparatus 10 when heating apparatus 10 is operatively associated with the first and second transparent bioreactor vessels 72.

The described methods are particularly advantageous in cultivating and promoting growth of bioreactor cultures requiring customized temperature regulation. Exemplary uses of this method may include bioprocessing of biopharmaceuticals and conducting bioprocessing experiments that are particularly sensitive to temperature conditions, require substantial temperature customization and regulation, when ideal temperature conditions are unknown/still being assessed/determined and when visual indicators are important in monitoring and managing bioprocessing.

Although certain methods may be described with reference to steps presented in a certain order, in many instances, these steps may be performed in any order as may be appreciated by one skilled in the art; the novel method is therefore not limited to the particular arrangement of steps disclosed herein

Although particular embodiments have been shown and described, it will be understood that they are not intended to limit the claims herein, and it will be obvious to those skilled in the art that various changes and modifications may be made without departing from the spirit and scope of the claims. The claims are intended to cover alternatives, modifications, and equivalent embodiments.

The foregoing disclosure has been presented for the purpose of illustration and description only and is not to be construed as limiting the claim scope in any way.

### REFERENCE NUMERALS

10 Heating apparatus
20 Flexible body of the heating apparatus
22 First layer of the flexible body
24 Second layer of the flexible body
26 Fastener, Fastener section of the flexible body
28 Cutout of flexible body
30 Transparent portions of the flexible body
40 Heating elements
50 Controller
60 Temperature sensor
62 Connector
70 Bioreactor
71 Bioreactor lid
72 Bioreactor vessel
74 Bioreactor agitator
75 Bioreactor spin filter
76 Bioreactor dip tube
78 Bioreactor sensor
80 Bioreactor system
82 User interface/display
84 Supplemental heater

1. A heating apparatus for a bioreactor, wherein the heating apparatus comprises:
a flexible body that is transparent, having a transparent portion comprising a material with translucent properties, and detachably couplable to and configured to be positioned about and substantially enclose sidewalls defining a perimeter of a transparent bioreactor vessel for containing a bioreactor culture;
a heating element coupled to the flexible body and arranged to transfer heat to the exterior sidewalls of the bioreactor vessel to uniformly heat the bioreactor culture, wherein the heating element is positioned throughout and spanning the extent of a heat transfer region of the flexible body, including the transparent portion, the heating element being configured as an electrically conductive, resistive wire, arranged in such a way that visibility through the transparent portion is not substantially obstructed, wherein the heating element is connectable to a controller for regulating heat transfer; and
a sensor coupled to the flexible body to detect a temperature and/or a change in temperature of the heating apparatus and/or the bioreactor culture, wherein the sensor is connectable to the controller to monitor and/or regulate a temperature of the heating apparatus and/or bioreactor culture.

2. The apparatus of claim 1, wherein the sensor and the heating element are configured as an integrated unit or component of the heating apparatus.

3. The apparatus of claim 1, wherein the heating element comprises one or more electrically conductive wires forming a heat transfer portion of the flexible body, wherein the heat transfer portion of flexible body is transparent and the heating element is configured and arranged so as not to substantially obstruct visibility of the bioreactor culture through the heat transfer portion when the heating apparatus is coupled to the transparent bioreactor vessel.

4. The apparatus of claim 1, wherein the flexible body allows for visibility of the bioreactor culture through at least one or more opposing exterior sidewalls of the flexible body when positioned about and adjoining opposing exterior side walls of the transparent bioreactor vessel.

5. The apparatus of claim 1, wherein the heating apparatus is configured to be reusably and removably coupled to a first bioreactor and is configured to perform a first heating action adapted for the first bioreactor;
wherein the heating apparatus is configured to be reusably and removably coupled to a second bioreactor and is configured to perform a second heating action adapted for the second bioreactor; and
wherein the first heating action is different than the second heating action.

6. The apparatus of claim 5, wherein the first and second heating actions are selected from the group consisting of: heating to a predetermined temperature or temperature range; transferring heat at a predetermined temperature; transferring heat at a predetermined heating rate; heating or limiting applied heat based on a temperature threshold; heating or limiting applied heat based on a change in temperature; heating for a predetermined duration; and/or applying heat in accordance with a predetermined program.

7. The apparatus of claim 5, wherein the first and second heating actions are different in view of and are dependent upon one or more factors selected from the group consisting of: a type of bioreactor vessel coupled to the heating apparatus; a configuration and/or material property of a bioreactor vessel couplable to the heating apparatus; an intended operation of the couplable bioreactor vessel; and/or a desired temperature, temperature range or change in temperature of a bioreactor culture of the bioreactor vessel coupled to the heating apparatus.

8. The apparatus of claim 5, wherein the first and second heating actions are transferring heat at a predetermined heating rate, wherein the first bioreactor is a single use bioreactor and the second bioreactor is a multi-use bioreactor, and wherein the heating apparatus is configured to heat the first transparent bioreactor vessel at a first predetermined rate and wherein the heating apparatus is configured to heat a second bioreactor vessel at a second predetermined rate higher than the first predetermined rate.

9. The apparatus of claim 1, wherein the flexible body is configured as a heating jacket and further comprises:
a first flexible layer comprising a first transparent portion allowing
for visibility through the first flexible layer; and
a second flexible layer comprising a second transparent portion
allowing for visibility through the second flexible layer,
wherein the heating element is positioned between the first and
second flexible layers, and wherein the bioreactor culture is viewable through the first and second transparent portions of the flexible body when the heating apparatus is coupled to the transparent bioreactor vessel.

10. The apparatus of claim 1, wherein the heating element is densely arranged in a series of closely positioned rows, columns and/or convolutions throughout the heat transfer portion of the flexible body.

11. A bioreactor system comprising:
a heating apparatus comprising:
a flexible body that is transparent, having a transparent portion comprising a material with translucent properties, and detachably couplable to and configured to be positioned about and substantially enclose sidewalls defining a perimeter of a transparent bioreactor vessel for containing a bioreactor culture;
a heating element coupled to the flexible body and arranged to transfer heat to the exterior sidewalls of the bioreactor vessel to uniformly heat the bioreactor culture, wherein the heating element is positioned throughout and spanning the extent of a heat transfer region of the flexible body, including the transparent portion, the heating element being configured as an electrically conductive, resistive wire, arranged in such a way that visibility through the transparent portion is not substantially obstructed, wherein the heating element is connectable to a controller for regulating heat transfer; and
a sensor coupled to the flexible body to detect a temperature and/or a change in temperature of the heating apparatus and/or the bioreactor culture, wherein the sensor is connectable to the controller to monitor and/or regulate a temperature of the heating apparatus and/or bioreactor culture; and
a controller for monitoring and/or regulating heat transfer, wherein the controller is connected to the heating element and the sensor.

12. The bioreactor system of claim 11 further comprising:
a single use bioreactor comprising a plastic bioreactor vessel; and
a multi-use bioreactor comprising a glass bioreactor vessel,
wherein the heating apparatus is configured to be removably coupled to and operate in connection with the plastic bioreactor vessel and the glass bioreactor vessel and wherein the heating apparatus is configured to perform a first heating action to the plastic bioreactor vessel and perform a second heating action to the glass bioreactor vessel, wherein the first heating action is different from the second heating action.

13. The bioreactor system of claim 11 further comprising:
a user interface operatively associated with the controller to allow a user to adjust a heating action of the heating apparatus.

14. The bioreactor system of claim 11 further comprising:
a supplemental heater connected to the controller and coupled to the transparent bioreactor vessel for transferring heat to the transparent bioreactor vessel, wherein the controller is configured to coordinate and centrally regulate heat transfer through heating apparatus and supplemental heater.

15. The bioreactor system according to claim 11, comprising:
a first bioreactor for cultivating organisms, wherein the first bioreactor comprises a first transparent bioreactor vessel containing a first bioreactor culture;
wherein the heating apparatus is operatively associated with the first transparent bioreactor vessel, wherein the heating apparatus is positioned about an exterior surface of the first bioreactor vessel or a bioreactor vessel component, wherein the heating apparatus transfers heat to the bioreactor culture, detects a temperature of the heating apparatus and/or the bioreactor culture, and is configured and arranged so as not to substantially obstruct visibility of the bioreactor culture; and
wherein the controller is connected to the heating apparatus for regulating a temperature of the bioreactor culture and heat applied by the heating apparatus,
wherein the heating apparatus is configured to perform a first heating action specific to and adapted for one or more parameters or conditions of the first bioreactor and is configured to perform a second heating action specific to and adapted for one or more parameters of a second bioreactor, wherein the first and second heating actions are different.

16. The system of claim 15, wherein the system further comprises a supplemental heater coupled to and configured to transfer heat to the first transparent bioreactor vessel, wherein the controller is connected to the supplemental heater and is configured to centrally control and coordinate heat applied by the heating apparatus and the supplemental heater in order to regulate the temperature of the heating apparatus and/or the first bioreactor culture.

17. The system of claim 15, further comprising the second bioreactor; wherein the first bioreactor is a single use bioreactor and the first transparent bioreactor vessel is a plastic transparent bioreactor vessel; and wherein the second bioreactor is a multi-use bioreactor comprising a glass bioreactor vessel; and wherein the heating apparatus is configured to be removably coupled to and operate in connection with the first and second transparent bioreactor vessels.

18. The system of claim 15, wherein the heating apparatus comprises:
a flexible body comprising one or more transparent portions allowing
for visibility of the first bioreactor culture when the heating apparatus is coupled to the portion of the bioreactor;
a heating element configured and arranged so as not to substantially
obstruct visibility of the bioreactor culture through the one or more transparent portions of the flexible body when the heating apparatus is coupled to the portion of the first bioreactor; and
a temperature sensor connected to the controller, wherein the temperature-sensor detects a temperature or change in temperature of the flexible body and/or the first bioreactor culture.

19. The system of claim 15, wherein the heating apparatus is integrally formed with, coupled to, attached to or connected to a component of the bioreactor selected from the group consisting of: a transparent bioreactor vessel wall; an impeller of the bioreactor; an impeller shaft of the bioreactor; a diptube of the bioreactor; and/or a spin filter of the bioreactor.

20. A bioreactor temperature regulating method utilizing a bioreactor heating apparatus with a bioreactor, the bioreactor comprising a transparent bioreactor vessel containing a bioreactor culture, and the bioreactor heating apparatus comprising:
a flexible body that is transparent, having a transparent portion comprising a material with translucent properties, and detachably couplable to and configured to be positioned about and substantially enclose sidewalls defining a perimeter of the transparent bioreactor vessel;
a heating element coupled to the flexible body and arranged to transfer heat to the exterior sidewalls of the bioreactor vessel to uniformly heat the bioreactor culture, wherein the heating element is positioned throughout and spanning the extent of a heat transfer region of the flexible body, including the transparent portion, the heating element being configured as an electrically conductive, resistive wire, arranged in such a way that visibility through the transparent portion is not substantially obstructed, wherein the heating element is connectable to a controller for regulating heat transfer; and
a sensor coupled to the flexible body to detect a temperature and/or a change in temperature of the heating apparatus and/or the bioreactor culture, wherein the sensor is connectable to the controller to monitor and/or regulate a temperature of the heating apparatus and/or bioreactor culture, the bioreactor regulating method comprising the steps of:
operatively associating the heating apparatus with the bioreactor vessel; and
applying heat to the bioreactor vessel or a bioreactor culture contained within the bioreactor vessel using the heating apparatus.

21. The method according to claim 20, further comprising:
detecting a condition, parameter or change thereof of the bioreactor culture and/or the heating apparatus using the heating apparatus; and
regulating a temperature of the bioreactor culture and/or heating apparatus based on the detected condition, parameter or change thereof of the bioreactor culture and/or the heating apparatus.

22. The method according to claim 20, further comprising the steps of:
operatively associating a heating apparatus with a first bioreactor vessel, wherein the heating apparatus has a transparent body;
performing a first heating action adapted for the first bioreactor vessel or
a first bioreactor culture contained therein using the heating apparatus;
operatively associating a heating apparatus with a second bioreactor
vessel;
performing a second heating action adapted for the second bioreactor
vessel or a second bioreactor culture contained therein using the heating apparatus, wherein the first and second heating actions are different; and
directly visualizing the first bioreactor cultures through the transparent body when heating apparatus is operatively associated with the first bioreactor and directly visualizing the second bioreactor culture through the transparent body when heating apparatus is operatively associated with the second bioreactor.

23. The method according to claim 20, further comprising the step of:
directly visualizing the first bioreactor culture through the transparent body of the heating apparatus and the transparent bioreactor vessel wall.
